(19) United States
(12) Patent Application Publication
(10) Pub. No.: US 2015/0258084 A1
(43) Pub. Date: Sep. 17, 2015

(54) STEM CELL COMPOSITIONS AND METHODS OF THEIR USE

(71) Applicant: Banner Health, Phoenix, AZ (US)

(72) Inventor: Mohamed A. Gaballa, Peoria, AZ (US)

(21) Appl. No.: 14/645,422

(22) Filed: Mar. 12, 2015

Related U.S. Application Data
(60) Provisional application No. 61/951,539, filed on Mar. 12, 2014.

(51) Int. Cl. A61K 31/4725; A61K 45/06; A61K 31/4439; A61K 35/34

(52) U.S. Cl. CPC A61K 31/4725; A61K 35/34; A61K 45/06; A61K 31/4439

(57) ABSTRACT

The present invention relates to admixtures of stem cells and TGF-β inhibitors. The invention provides methods for characterizing, isolating, and culturing stem cells from human tissue samples. The invention also provides compositions and methods useful for treating subjects with disease (e.g. heart disease).

### RELATED APPLICATIONS

This application claims priority to U.S. Provisional Patent Application Ser. No. 61/951,539, filed on Mar. 12, 2014, which is hereby incorporated by reference herein in its entirety.

### STATEMENT OF RIGHTS UNDER FEDERALLY-SPONSORED RESEARCH

This invention was made with government support under NIH RO1 AG027263, awarded by the National Institute of Aging. The government has certain rights in this invention.

### FIELD OF THE INVENTION

The present invention relates to admixtures of stem cells and TGF-β inhibitors. The invention provides methods for characterizing, isolating, and culturing stem cells from human tissue samples. The invention also provides compositions and methods useful for treating subjects with disease (e.g. heart disease).

### BACKGROUND OF THE INVENTION

Stem cells derived from a human subject are potentially useful for a variety purposes, including regeneration of damaged tissues, reproduction, and as cellular models that could inform personal medicine, including diagnoses, treatments to alleviate a condition of disease or disorder, or warnings of adverse reaction to a potential treatment. Currently, induced pluripotent stem cells (iPS cells) are the dominant model system. iPS cells are derived from dividing multipotent or committed cells (such as fibroblasts, fat stem cells, lymphocytes) by the introduction of different combinations of specific transcription factors involved in regulating pluripotency (e.g., OCT4, SOX2, NANOG, KLF4, MYC, LIN28, TERT). The transcription factor levels are increased by a variety of mechanisms, including viral reprogramming of the cells' DNA, and the direct introduction into the cell of pluripotency proteins or of mRNA encoding for the pluripotency proteins. The iPS cells generated through these methods are extraordinarily similar to embryonic stem cells (ES cells), including the capacity to differentiate into cells from all three germ layers, gene expression profiles, and capacity to form teratomas when injected into animals. The iPS cells offer the advantage over ES cells of being from the organism of interest, that is, they are autologous. However, obstacles to clinical use of iPS cells include that iPS cells may be prone to cancer or other pathologies, that the iPS cells, as “artificial”, may not faithfully recapitulate disease processes (e.g., due to epigenetic factors), and that generation of iPS cells is relatively expensive and time consuming.

Thus, there is a need in the art for methods to identify and directly harvest autologous pluripotent stem cells that reduce or avoid the aforementioned limitations, from a human subject. The present invention meets this need by providing isolated stem cells. The present invention further provides compositions and methods useful for treating diseases including, for example, heart diseases.

### SUMMARY OF THE INVENTION

The present invention provides admixtures of stem cells and TGF-β inhibitors. The invention further provides methods for characterizing, isolating, and culturing stem cells from human tissue samples. The invention also provides compositions and methods useful for treating subjects with disease such as, for example, heart disease.

In some embodiments, the invention provides a composition comprising a human stem cell in admixture with a TGFβ inhibitor. In some aspects, the human stem cell in the admixture is an iPS cell, an embryonic stem cell, a stem cell line, a pluripotent stem cell, a multipotent stem cell, or an adult stem cell. In some embodiments, the TGF-β inhibitor is a small molecule, an antibody, or a nucleic acid. In certain embodiments, the small molecule is SB431542 or SIS3. In some embodiments, the human stem cell in the admixture has increased pluripotency marker expression levels compared to a corresponding human stem cell not in an admixture with the TGF-β inhibitor. In some embodiments, the human stem cell in the admixture has an enhanced differentiation potential compared to a corresponding human stem cell not in an admixture with the TGF-β inhibitor. In yet other embodiments, the human stem cell in the admixture has a faster growth rate compared to the growth rate of a corresponding human stem cell not in an admixture with the TGF-β inhibitor. In some embodiments, the growth rate is assessed by measuring the increase in the number or percentage of c-Kit+ cells. In certain embodiments, the human stem cell is a cardiac stem cell. In other embodiments, the human stem cell expresses c-Kit and Nanog but not KDR. In yet other embodiments, the human stem cell further expresses one or more markers selected from the group consisting of Sox2, Hey1, SMA, Vimentin, Cyclin D2, Snail, and E-cadherin, and not one or more of the markers selected from the group consisting of CD34 and CD45. In some embodiments, the human stem cell is obtained from a subject having or suspected of having a heart disease.

In some embodiments, the invention provides methods for increasing the pluripotency of a human stem cell comprising treating a human stem cell with an effective amount of a TGF-β inhibitor. In some embodiments, the TGF-β inhibitor is a small molecule, an antibody, or a nucleic acid. In other embodiments, the small molecule is SB431542 or SIS3. In yet other embodiments, the human stem cell in the admixture is an iPS cell, an embryonic stem cell, a stem cell line, a pluripotent stem cell, a multipotent stem cell, or an adult stem cell. In other embodiments, the human stem cell is a cardiac stem cell. In some embodiments, the human stem cell is obtained from a subject having or suspected of having a heart disease. In yet other embodiments, the human stem cell expresses c-Kit and Nanog but not KDR. In still other embodiments, the human stem cell further expresses one or more markers selected from the group consisting of Sox2, Hey1, SMA, Vimentin, Cyclin D2, Snail, and E-cadherin, and not one or more of the markers selected from the group consisting of CD34 and CD45.

In some embodiments, the present invention provides methods for increasing the differentiation potential of a human stem cell comprising treating a human stem cell with an effective amount of a TGF-β inhibitor. In certain embodiments, the TGF-β inhibitor is a small molecule, an antibody, or a nucleic acid. In other embodiments, the small molecule is SB431542 or SIS3. In some embodiments, the human stem cell in the admixture is an iPS cell, an embryonic stem cell, a stem cell line, a pluripotent stem cell, a multipotent stem cell, or an adult stem cell. In yet other embodiments, the human stem cell is a cardiac stem cell. In other embodiments, the human stem cell is obtained from a subject having or suspected of having a heart disease. In some embodiments, the human stem cell expresses c-Kit and Nanog but not KDR. In certain embodiments, the human stem cell further expresses one or more markers selected from the group consisting of Sox2, Hey1, SMA, Vimentin, Cyclin D2, Snail, and E-cadherin, and not one or more of the markers selected from the group consisting of CD34 and CD45.

In some embodiments, the present invention provides methods for enhancing the growth rate of a human stem cell comprising treating a human stem cell with an effective amount of a TGF-β inhibitor. In certain embodiments, the TGF-β inhibitor is a small molecule, an antibody, or a nucleic acid. In other embodiments, the small molecule is SB431542 or SIS3. In yet other embodiments, the human stem cell in the admixture is an iPS cell, an embryonic stem cell, a stem cell line, a pluripotent stem cell, a multipotent stem cell, or an adult stem cell. In some embodiments, the human stem cell is a cardiac stem cell. In other embodiments, the human stem cell is obtained from a subject having or suspected of having a heart disease. In yet other embodiments, the human stem cell expresses c-Kit and Nanog but not KDR. In still other embodiments, the human stem cell further expresses one or more markers selected from the group consisting of Sox2, Hey1, SMA, Vimentin, Cyclin D2, Snail, and E-cadherin, and not one or more of the markers selected from the group consisting of CD34 and CD45.

In some embodiments, the present invention provides methods of treating a subject suffering from a disease, comprising administering to the subject an effective amount of human stem cells, wherein the human stem cells are pretreated with a TGF-β inhibitor. In other embodiments, the subject has a heart disease. In some embodiments, the subject has a heart disease selected from the group consisting of chronic heart failure, myocardial infarction, congestive heart failure, congenital heart disease, cardiomyopathy, pericarditis, angina, and coronary artery disease. In yet other embodiments, the human stem cells are iPS cells, embryonic stem cells, a stem cell line, pluripotent stem cells, multipotent stem cells, or adult stem cells. In other embodiments, the TGF-β inhibitor is a small molecule, an antibody, or a nucleic acid. In some embodiments, the small molecule is SB431542 or SIS3. In yet other embodiments, the human stem cells are cardiac stem cells. In other embodiments, the human stem cells express c-Kit and Nanog but not KDR. In yet other embodiments, the human stem cells further expresses one or more markers selected from the group consisting of Sox2, Hey1, SMA, Vimentin, Cyclin D2, Snail, and E-cadherin, and not one or more of the markers selected from the group consisting of CD34 and CD45.

These and other embodiments of the present invention will readily occur to those of skill in the art in light of the disclosure herein, and all such embodiments are specifically contemplated.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

### BRIEF DESCRIPTION OF THE DRAWINGS

FIGS. 1A and 1B set forth data showing characterization data for explant-derived stem cells from sham and CHF animals.

FIG. 2 sets forth data showing TGF-β levels for sham and CHF-derived c-Kit+ cells.

FIGS. 3A, 3B, and 3C set forth data showing expression levels of various markers in sham and CHF-derived c-Kit+ cells.

FIGS. 4A and 4B set forth data showing expression levels of various markers in sham and CHF-derived c-Kit+ cells that were treated in culture with TGF-β inhibitors.

FIGS. 5A and 5B set forth data showing Nanog protein levels in both sham and CHF cardiac stem cells that were treated in culture with TGF-β inhibitors.

FIGS. 6A and 6B set forth data showing Nanog protein levels in both sham and CHF cardiac stem cells that were treated in culture with TGF-β inhibitors.

FIG. 7 sets forth data showing differentiation potential of sham compared with CHF cardiac stem cells that were treated in culture with TGF-β inhibitors.

FIGS. 8A, 8B, and 8C set forth data showing cell growth of sham control compared with CHF cardiac stem cells that were treated in culture with TGF-β inhibitors.

FIGS. 9A, 9B, 9C, and 9D set forth data showing FACS analysis of sham or CHF explant-derived cells in the presence of TGF-β inhibitors.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present invention relates, in part, to the discovery that TGF-β inhibition enhances various properties of isolated stem cells. The present invention provides admixtures of stem cells and TGF-β inhibitors. The invention also provides methods for characterizing, isolating, and culturing stem cells from human tissue samples. The invention further provides compositions and methods useful for treating subjects with disease (e.g. heart disease).

The section headings are used herein for organizational purposes only, and are not to be construed as in any way limiting the subject matter described herein.

### Stem Cells

Stem cells are undifferentiated cells defined by their ability at the single cell level to both self-renew and differentiate to produce progeny cells, including self-renewing progenitors, non-renewing progenitors, and terminally differentiated cells. Stem cells, depending on their level of differentiation, are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, which is able to give rise to all embryonic and extraembryonic cell types; (2) pluripotent, which is able to give rise to all embryonic cell types. i.e., endoderm, mesoderm, and ectoderm; (3) multipotent, which is able to give rise to a subset of cell lineages, but all within a particular tissue, organ, or physiological system (for example, hematopoietic stem cells (HSC) can produce progeny that include HSC (self-renewal), blood cell restricted oligopotent progenitors and the cell types and elements (e.g., platelets) that are normal components of the blood); (4) oligopotent, which is able to give rise to a more restricted subset of cell lineages than multipotent stem cells; and (5) unipotent, which is able to give rise to a single cell lineage (e.g., spermatogenic stem cells).

Stem cells useful in the compositions and methods of the present invention include embryonic cells of various types, exemplified by human embryonic stem (hES) cells, described by Thomson et al. (1998) Science 282:1145; U.S. Pat. No. 7,615,374; U.S. Pat. No. 7,611,852; U.S. Pat. No. 7,582,479; U.S. Pat. No. 7,514,260; U.S. Pat. No. 7,439,064, U.S. Pat. No. 7,390,657; U.S. Pat. No. 7,220,584; U.S. Pat. No. 7,217,569; U.S. Pat. No. 7,148,062; U.S. Pat. No. 7,029,913; U.S. Pat. No. 6,887,706; U.S. Pat. No. 6,613,568; U.S. Pat. No. 6,602,711; U.S. Pat. No. 6,280,718; U.S. Pat. No. 6,200,806; and U.S. Pat. No. 5,843,780, each of which is herein incorporated in their entirety by reference; and human embryonic germ (hEG) cells (Shambloft et al., Proc. Natl. Acad. Sci. USA 95:13726, 1998). Other useful stem cells are lineage committed stem cells, such as hematopoietic or pancreatic stem cells. Examples of multipotent cells useful in methods provided herein include, but are not limited to, human umbilical vein endothelial (HuVEC) cells, human umbilical artery smooth muscle (HuASMC) cells, human differentiated stem (HKB-II) cells, cardiac stem cells, and human mesenchymal stem (hMSC) cells.

Adult stem cells are generally limited to differentiating into different cell types of their tissue of origin. However, if the starting stem cells are derived from the inner cell mass of the embryo, they can give rise to all cell types of the body derived from the three embryonic germ layers: endoderm, mesoderm and ectoderm. Stem cells with this property are said to be pluripotent. Embryonic stem cells are one kind of pluripotent stem cell. Somatic stem cells have major advantages, for example, using somatic stem cells allows a patient's own cells to be expanded in culture and then re-introduced into the patient. Of course, induced pluripotent stem cells (iPS cells) from a patient provide a source of cells that can be expanded and re-introduced to the patient, before or after stimulation to differentiate to a desired lineage of phenotype.

Cells derived from embryonic sources can include embryonic stem cells or stem cell lines obtained from a stem cell bank or other recognized depository institution. Other means of producing stem cell lines include the method of Chung et al (2006) which comprises taking a blastomere cell from an early stage embryo prior to formation of the blastocyst (at around the 8-cell stage). The technique corresponds to the pre-implantation genetic diagnosis technique routinely practiced in assisted reproduction clinics. The single blastomere cell is then co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli.

In one embodiment, the stem cell is isolated. Most conventional methods to isolate a particular stem cell of interest involve positive and negative selection using markers of interest. Agents can be used to recognize stem cell markers, for instance labeled antibodies that recognize and bind to cell-surface markers or antigens on desired stem cells. Antibodies or similar agents specific for a given marker, or set of markers, can be used to separate and isolate the desired stem cells using fluorescent activated cell sorting (FACS), panning methods, magnetic particle selection, particle sorter selection and other methods known to persons skilled in the art, including density separation (Xu et al. (2002) Circ. Res. 91:501; U.S. patent application Ser. No. 20030022367) and separation based on other physical properties (Doevendans et al. (2000) J. Mol. Cell. Cardiol. 32:839-851). Alternatively, genetic selection methods can be used, where a stem cell can be genetically engineered to express a reporter protein operatively linked to a tissue-specific promoter and/or a specific gene promoter; therefore the expression of the reporter can be used for positive selection methods to isolate and enrich the desired stem cell. For example, a fluorescent reporter protein can be expressed in the desired stem cell by genetic engineering methods to operatively link the marker protein to a promoter active in a desired stem cell (Klug et al. (1996) J. Clin. Invest. 98:216-224; U.S. Pat. No. 6,737,054). Other means of positive selection include drug selection, for instance as described by Klug et al., supra, involving enrichment of desired cells by density gradient centrifugation. Negative selection can be performed, selecting and removing cells with undesired markers or characteristics, for example fibroblast markers, epithelial cell markers etc.

Undifferentiated ES cells express genes that can be used as markers to detect the presence of undifferentiated cells. The polypeptide products of such genes can be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. Undifferentiated human ES cell lines do not stain for SSEA-1, but differentiated cells stain strongly for SSEA-1. Methods for proliferating hES cells in the undifferentiated form are described in WO 99/20741, WO 01/51616, and WO 03/020920, which are herein incorporated by reference in their entirety.

In one embodiment, the methods provide for enrichment and isolation of stem cells. The stem cells are selected for a characteristic of interest. In some embodiments, a wide range of markers may be used for selection. One of skill in the art will be able to select markers appropriate for the desired cell type. The characteristics of interest include expression of particular markers of interest, for example specific subpopulations of stem cells and stem cell progenitors will express specific markers. In some embodiments, stem cells of the present invention are selected using one or more markers selected from the list consisting of c-Kit, Nanog, KDR, Sox2, Hey1, SMA, Vimentin, Cyclin D2, Snail, and E-cadherin, CD34, and CD45.

In one embodiment, the stem cells are expanded. The cells are optionally collected, separated, and further expanded, generating larger populations of stem cells for use in making cells of a particular cell type or cells having an enhanced efficiency of homologous recombination.

### Induced Pluripotent Stem Cells (iPS Cells)

The production of iPS cells is generally achieved by the introduction of nucleic acid sequences encoding stem cell-associated genes into an adult, somatic cell. Historically, these nucleic acids have been introduced using viral vectors and the expression of the gene products results in cells that are morphologically, biochemically, and functionally similar to pluripotent stem cells (e.g., embryonic stem cells). This process of altering a cell phenotype from a somatic cell phenotype to a pluripotent stem cell phenotype is termed reprogramming iPS cells can be generated or derived from terminally differentiated somatic cells, as well as from adult stem cells. That is, a non-pluripotent stem cell can be rendered pluripotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors. Reprogramming can be achieved by introducing a combination of stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Ten, and LIN28. In one embodiment, successful reprogramming is accomplished by introducing Oct-4, a member of the Sox family, a member of the Klf family, and a member of the Myc family to a somatic cell. In one embodiment each of Oct 4, Sox2, Nanog, c-MYC and Klf4 are used to reprogram a human stem cell.

The efficiency of reprogramming (i.e., the number of reprogrammed cells) derived from a population of starting cells can also be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135, which are incorporated herein by reference in their entirety. It is contemplated that the methods described herein can also be used in combination with a single small molecule (or a combination of small molecules) that enhances the efficiency of induced pluripotent stem cell production or that replaces one or more reprogramming factors during the reprogramming process. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), and trichostatin (TSA), among others.

To confirm the induction of pluripotent stem cells, isolated clones can be tested for the expression of a stem cell marker. Such expression identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Natl. Methods for detecting the expression of such markers can include, for example, RT-PCR and immunological methods that detect the presence of the encoded polypeptides. In one embodiment, detection does not involve RT-PCR, but rather focuses on detection of protein markers.

The pluripotent stem cell character of the isolated cells can be confirmed by any of a number of tests evaluating the expression of ES markers and the ability to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers further indicates that the cells are pluripotent stem cells.

### TGF-β Inhibitors

A TGF-β inhibitor is a compound that inhibits TGF-β signal transduction by inhibiting any of the factors constituting the TGF-β signal transduction system pathway, such as TGF-β ligand, TGF-β Type I receptors, TGF-β Type II receptors, TGF-β Type III receptors (β-glycan and endoglin), soluble forms of the TGF-β receptors, Smad proteins (1-8), antibodies against receptors and ligands implicated in the signaling pathway, nucleic acid based molecules (e.g., antisense, siRNA, aptamers and ribozymes) targeting the pathway members, or a combination thereof.

An inhibitor of a TGFβR can function in a competitive or non-competitive manner, and can function, in one embodiment, by interfering with the expression of the TGFβR polypeptide. A TGFβR inhibitor includes any chemical or biological entity that, upon treatment of a cell, results in inhibition of a biological activity caused by activation of the TGFβR in response to binding of its natural ligand. While any TGF-β signaling pathway inhibitor can potentially be used in the methods described herein, it is preferable that a TGF-β signaling pathway inhibitor is either selective for, or specific for, a member of the TGF-β signaling pathway. Specific is the dose necessary for the inhibiting agent to inhibit the TGF-β signaling pathway, the inhibiting agent does not have any other substantial pharmacological action in the cell or host. Selective is the dose of the inhibitor necessary for inhibition of the TGF-β signaling pathway is at least 2-fold lower than the dose necessary for activation or inhibition of another pharmacological action as measured by the ED50 or EC50 of the agent for each pharmacological effect; preferably the dose of inhibitor necessary for TGF-β pathway inhibition is at least 5-fold lower, at least 10 fold lower, at least 20-fold lower, at least 30-fold lower, at least 40-fold lower, at least 50-fold lower, at least 60-fold lower, at least 70-fold lower, at least 80-fold lower, at least 90-fold lower, at least 100-fold lower, at least 500-fold lower, at least 1000 fold lower or more, than the dose necessary for another pharmacological action. Thus, to be clear, the agents useful for the methods described herein primarily inhibit the TGF-β signaling pathway with only minor, if any, effects on other pharmacological pathways, and the dose used for inhibition of the TGF-β signaling pathway is sub-clinical or sub-threshold for other pharmacological responses.

Some non-limiting examples of small molecule inhibitors of TGFβRs include 24346-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole, which can be purchased from Calbiochem (San Diego, Calif.). Other small molecule inhibitors include, but are not limited to, SB-431542 (see e.g., Halder et al., 2005; Neoplasia 7(5):509-521), SM16 (see e.g., Fu, K et al., 2008; Arteriosclerosis, Thrombosis and Vascular Biology 28(4):665), SIS3, and SB-505124 (see e.g., Dacosta Byfield, S., et al., 2004; Molecular Pharmacology 65:744-52), among others.

Inhibitors of TGF-β signaling are described in Callahan, J. F. et al., J. Med. Chem. 45, 999-1001 (2002); Sawyer, J. S. et al., J. Med. Chem. 46, 3953-3956 (20031; Gellibert, F. et al., J. Med. Chem. 47, 4494-4506 (2004); Tojo, M. et al., Cancer Sci. 96: 791-800 (2005); Valdimarsdottir, G. et al., APMIS 113, 773-389 (2005); Petersen et al. Kidney International 73, 705-715 (2008); Yingling, J. M. et al., Nature Rev. Drug Disc. 3, 1011-1022 (2004); Byfield, S. D. et al., Mol. Pharmacol., 65, 744-752 (2004); Dumont, N, et al., Cancer Cell 3, 531-536 (2003); WO Publication No. 2002/094833; WO Publication No. 2004/026865; WO Publication No. 2004/067530; WO Publication No. 209/032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031; U.S. Pat. No. 6,419,928; U.S. Pat. No. 7,030,125; U.S. Pat. No. 6,943,191; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0138188 and U.S. Publication No. 2009/0036382, the contents of each which are herein incorporated by reference in their entirety.

Oligonucleotide based modulators of TGF-β signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, each of which are herein incorporated by reference in their entirety. Other antisense nucleic acids and siRNAs can be obtained by methods known to one of ordinary skill in the art.

Antibodies that can be used according to the methods described herein include complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone).

Antibodies for use in the methods described herein can be obtained from commercial sources such as AbCam (Cambridge, Mass.), New England Biolabs (Ipswich, Mass.), Santa Cruz Biotechnologies (Santa Cruz, Calif.), Biovision (Mountain View, Calif.), R&D Systems (Minneapolis, Minn.), and Cell Signaling (Danvers, Mass.), among others. Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., Eds., Antibodies: A Laboratory Manual, Cold Spring Harbor Laboratory, New York (1988), which is hereby incorporated by reference in its entirety.

siRNAs useful for targeting TGFβR expression can be readily designed and tested. Chalk et al. (Nucl. Acids Res. 33: D131-D134 (2005)) describe a database of siRNA sequences and a predictor of siRNA sequences. Linked to the sequences in the database is information such as siRNA thermodynamic properties and the potential for sequence-specific off-target effects. The database and associated predictive tools enable the user to evaluate an siRNA's potential for inhibition and non-specific effects.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al., Nature 411:494-498 (2001); Elbashir, S. M., et al., Genes & Development 15:188-200 (2001); Harborth, J. et al., J. Cell Science 114:4557-4565 (2001); Masters, J. R. et al., Proc. Natl. Acad. Sci., USA 98:8012-8017 (2001); and Tuschl, T. et al., Genes & Development 13:3191-3197 (1999)). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al., Genes Dev. 16:948-958 (2002); McManus, M. T. et al., RNA 8:842-850 (2002); Paul, C. P. et al., Nat. Biotechnol. 20:505-508 (2002); Miyagishi, M. et al., Nat. Biotechnol. 20:497-500 (2002); Sui, G. et al., Proc. Natl. Acad. Sci., USA 99:5515-5520 (2002); Brummelkamp, T. et al., Cancer Cell 2:243 (2002); Lee, N. S., et al., Nat. Biotechnol. 20:500-505 (2002); Yu, J. Y., et al., Proc. Natl. Acad. Sci., USA 99:6047-6052 (2002); Zeng, Y., et al., Mol. Cell. 9:1327-1333 (2002); Rubinson, D. A., et al., Nat. Genet. 33:401-406 (2003); Stewart, S. A., et al., RNA 9:493-501 (2003)).

### Tissue Samples

In some embodiments, stem cells of the present invention are isolated from human tissues. Any bodily tissue may be used in the methods of the present invention, including, for example, tissue from an organ, from skin, from adipose, and from blood. Organ tissues useful for the compositions and methods of the present invention include liver, lung, heart, kidney, heart, brain, and pancreas. In certain embodiments, stem cells are isolated from heart tissue from atrial or ventricular biopsy specimens from human subjects. Such subjects may have heart disease including, for example, myocardial infarction or chronic heart failure.

### Isolation and Maintenance of Stem Cells

In some embodiments, stem cells are isolated from a sample or biopsy of bodily tissue by digested by enzymatic digestion, mechanical separation, filtration, centrifugation and combinations thereof. The number and quality of the isolated stem cells can vary depending, e.g., on the quality of the tissue used, the compositions of perfusion buffer solutions, and the type and concentration of enzyme. Frequently used enzymes include, but are not limited to, collagenase, pronase, trypsin, dispase, hyaluronidase, thermolysin and pancreatin, and combinations thereof. Collagenase is most commonly used, often prepared from bacteria (e.g. from Clostridium histolyticum), and may often consist of a poorly purified blend of enzymes, which may have inconsistent enzymatic action. Some of the enzymes exhibit protease activity, which may cause unwanted reactions affecting the quality and quantity of viable/healthy cells. It is understood by those of skill in the art to use enzymes of sufficient purity and quality to obtain viable stem cell populations.

The methods of the invention comprise culturing the stem cells obtained from human tissue samples. In one embodiment, the populations of stem cells are plated onto a substrate. In the present invention, cells (e.g., stem cells) are plated onto a substrate which allows for adherence of cells thereto, i.e., a surface which is not generally repulsive to cell adhesion or attachment. This may be carried out, e.g., by plating the cells in a culture system (e.g., a culture vessel) which displays one or more substrate surfaces compatible with cell adhesion. When the said one or more substrate surfaces contact the suspension of cells (e.g., suspension in a medium) introduced into the culture system, cell adhesion between the cells and the substrate surfaces may ensue. Accordingly, the term “plating onto a substrate which allows adherence of cells thereto” refers to introducing cells into a culture system which features at least one substrate surface that is generally compatible with adherence of cells thereto, such that the plated cells can contact the said substrate surface. General principles of maintaining adherent cell cultures are well-known in the art.

As appreciated by those skilled in the art, the cells may be counted in order to facilitate subsequent plating of the cells at a desired density. Where, as in the present invention, the cells after plating may primarily adhere to a substrate surface present in the culture system (e.g., in a culture vessel), the plating density may be expressed as number of cells plated per mm² or cm² of the said substrate surface.

Typically, after plating of the stem cells of the present invention, the cell suspension is left in contact with the adherent surface to allow for adherence of cells from the cell population to the said substrate. In contacting the stem cells with adherent substrate, the cells may be advantageously suspended in an environment comprising at least a medium, in the methods of the invention typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

The medium may be a suitable culture medium as described elsewhere in this specification. Preferably, the composition of the medium may have the same features, may be the same or substantially the same as the composition of medium used in the ensuing steps of culturing the attached cells. Otherwise, the medium may be different.

Cells from the stem cell population or from tissue explants of the present invention, which have adhered to the said substrate, preferably in the said environment, are subsequently cultured for at least 7 days, for at least 8 days, or for at least 9 days, for at least 10 days, at least 11, or at least 12 days, at least 13 days or at least 14 days, for at least 15 days, for at least 16 days or for at least 17 days, or even for at least 18 days, for at least 19 days or at least 21 days or more. The term “culturing” is common in the art and broadly refers to maintenance and/or growth of cells and/or progeny thereof.

In some embodiments, the stem cells may be cultured for at least between about 10 days and about 40 days, for at least between about 15 days and about 35 days, for at least between about 15 days and 21 days, such as for at least about 15, 16, 17, 18, 19 or 21 days. In some embodiments, the stem cells of the invention may be cultured for no longer than 60 days, or no longer than 50 days, or no longer than 45 days.

The tissue explants and stem cells and the further adherent stem cells are cultured in the presence of a liquid culture medium. Typically, the medium will comprise a basal medium formulation as known in the art. Many basal media formulations can be used to culture the stem cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS, Lonza), 100 U/ml penicillin G, 100 μg/ml streptomycin and 2 mmol/L L-glutamine (Sigma-Aldrich). Other embodiments may employ further basal media formulations, such as chosen from the ones above.

For use in culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Also contemplated is supplementation of cell culture medium with mammalian plasma or sera. Plasma or sera often contain cellular factors and components that are necessary for viability and expansion. The use of suitable serum replacements is also contemplated (e.g., FBS).

In some embodiments, the medium comprises one or more TGF-β inhibitors, including, for example, SB431542 (Sigma-Aldrich) or SIS3 (Sigma-Aldrich).

As described, the present inventors have realized that by culturing tissue explants and stem cells for time durations as defined above, and preferably using media compositions as described above, a progenitor or stem cell of the invention emerges and proliferates. As detailed in the Examples section, the progenitor or stem cell may be distinguished from other cell types present in the primary cell culture by, among others, its expression of various markers.

The inventors also realized that the proliferation and enrichment of the culture for the said progenitor or stem cell may be further promoted by incubating a TGF-β inhibitor in the culture medium such that this enhances growth rate, differentiation potential, and pluripotency. In such conditions, the progenitor or stem cell of the invention can advantageously proliferate and become a prevalent cell type in the cell culture.

### Differentiation Potential of Stem Cells

In some embodiments, the methods of the present invention enhance the differentiation potential of stem cells isolated from a human tissue. Differentiation potential can be assessed by culturing stem cells and subsequently measuring the expression levels of a pluripotency marker. In one embodiment, cardiac stem cells are cultured and the expression levels of cardiac troponin T (TnT) are measured by immunohistochemistry to determine differentiation potential.

Identification and subsequent isolation of differentiated cells from their undifferentiated counterparts can be carried out by methods well known in the art. For example, cells that have been induced to differentiate can be identified by selectively culturing cells under conditions whereby differentiated cells outnumber undifferentiated cells. Similarly, differentiated cells can be identified by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. In addition, whole genome analysis using microarray technology can be used to identify differentiated cells.

### Growth Rates of Stem Cell In Vitro

In some embodiments, the methods of the present invention enhance the growth rate of human stem cells in vitro.

As described, the above culturing of tissue explants leads to the proliferation of stem cells of the invention in the culture. The said culturing can be advantageously continued until the stem cells of the invention have proliferated sufficiently. For example, the said culturing can be continued until the cell population achieved a certain degree of confluence, e.g., at least 40%, preferably at least 50%, more preferably at least 60% and even more preferably at least 70%, e.g., at least 80% or at least 90% or more confluence. The term “confluence” as used herein refers to a density of cultured cells in which the cells contact one another covering substantially all of the surfaces available for growth (i.e., fully confluent).

The method of the invention may thus provide for a cell population comprising a considerable fraction of stem cells and the fraction of the said stem cells can be increased by incubation with a TGF-β inhibitor. Typically, the population of c-Kit+ cells will comprise less than 10% of the said stem cells, but the inventors found that typically higher proportions of the said progenitor or stem cells will be obtained with TGF-β inhibitors, e.g., greater than 10%, greater than 15%, greater than 20%, greater than 25% or more. Moreover, the method may even yield a substantially homogeneous or homogeneous population of the said stem cells. The fraction of the stem cells can be evaluated by any appropriate standard method, e.g., by flow cytometry.

### Characterization of Stem Cells

In some embodiments, stem cells of the present invention are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. In certain embodiments, the marker proteins used to identify and characterize the stem cells are selected from the list consisting of c-Kit, Nanog, KDR, Sox2, Hey1, SMA, Vimentin, Cyclin D2, Snail, and E-cadherin, CD34, and CD45.

In some embodiments, methods of the present invention increase pluripotency markers in the isolated stem cells. Tissue explants are cultured in the presence of an effective amount of a TGF-β inhibitor. Following incubation, the expression levels of one or more pluripotency marker is determined in the stem cells.

### Subjects

In certain embodiments of all the above-described methods, the subject is a human subject. In certain embodiments, the subject is diagnosed with or suspected of having had a disease. In other embodiments, the patient is diagnosed with or suspected of having a heart disease, or is believed to have been exposed to or to be at risk for exposure to a heart disease. In some embodiments, the subject has a heart disease selected from the group consisting of chronic heart failure, myocardial infarction, congestive heart failure, congenital heart disease, cardiomyopathy, pericarditis, angina, and coronary artery disease.

### Methods of Treatment

The present invention provides methods of treating a disease in a subject, comprising administering to the subject an effective amount of human stem cells, wherein the human stem cells are pretreated with a TGF-β inhibitor. In one aspect, the human stem cells described herein can be produced from stem cells isolated from a subject having a disease. The isolated stem cells can be used to treat the disease by administering an effective amount of human stem cells to the subject. In some embodiments, an effective amount is a dosage is sufficient to generate significant numbers of new cardiomyocytes cells in the heart, and/or at least partially replace necrotic heart tissue, and/or produce a clinically significant change in heart function. A clinically significant improvement in heart performance can be determined by measuring the left ventricular ejection fraction, prior to, and after administration of cells, and determining at least a 5% increase, preferably 10% or more, in the total ejection fraction. Standard procedures are available to determine ejection fraction, as measured by blood ejected per beat. Dosages can vary from about 100-10⁷, 1000-10⁶ or 10⁴-10⁵ cells. The cells of the present invention can be administered either intravenously, intracoronary, or intraventricularly. A catheter can be used for the latter two routes of administration.

A wide range of diseases are recognized as being treatable with stem cell therapies. As non-limiting examples, these include disease marked by a failure of naturally occurring stem cells, such as aplastic anemia, Fanconi anemia, and paroxysmal nocturnal hemoglobinuria (PNH). Others include, for example: acute leukemias, including acute lymphoblastic leukemia (ALL), acute myelogenous leukemia (AML), acute biphenotypic leukemia and acute undifferentiated leukemia; chronic leukemias, including chronic myelogenous leukemia (CML), chronic lymphocytic leukemia (CLL), juvenile chronic myelogenous leukemia (JCML) and juvenile myelomonocytic leukemia (JMML); myeloproliferative disorders, including acute myelofibrosis, angiogenic myeloid metaplasia (myelofibrosis), polycythemia vera and essential thrombocythemia; lysosomal storage diseases, including mucopolysaccharidoses (MPS), Hurler's syndrome (MPS-IH), Scheie syndrome (MPS-IS), Hunter's syndrome (MPS-II), Sanfilippo syndrome (MPS-III), Morquio syndrome (MPS-IV), Maroteaux-Lamy Syndrome (MPS-VI), Sly syndrome, beta-glucuronidase deficiency (MPS-VII), adrenoleukodystrophy, mucolipidosis II (I-cell Disease), Krabbe disease, Gaucher's disease, Niemann-Pick disease, Wolman disease and metachromatic leukodystrophy; histiocytic disorders, including familial erythrophagocytic lymphohistiocytosis, histiocytosis-X and hemophagocytosis; phagocyte disorders, including Chediak-Higashi syndrome, chronic granulomatous disease, neutrophil actin deficiency and reticular dysgenesis; inherited platelet abnormalities, including amegakaryocytosis/congenital thrombocytopenia; plasma cell disorders, including multiple myeloma, plasma cell leukemia, and Waldenstrom's macroglobulinemia. Other malignancies treatable with stem cell therapies include but are not limited to breast cancer, Ewing sarcoma, neuroblastoma and renal cell carcinoma, among others. Also treatable with stem cell therapy are: lung disorders, including COPD and bronchial asthma; congenital immune disorders, including ataxia-telangiectasia, Kostmann syndrome, leukocyte adhesion deficiency, DiGeorge syndrome, bare lymphocyte syndrome, Omenn's syndrome, severe combined immunodeficiency (SCID), SCID with adenosine deaminase deficiency, absence of T & B cells SCID, absence of T cells, normal B cell SCID, common variable immunodeficiency and X-linked lymphoproliferative disorder; other inherited disorders, including Lesch-Nyhan syndrome, cartilage-hair hypoplasia, Glanzmann thrombasthenia, and osteopetrosis; neurological conditions, including acute and chronic stroke, traumatic brain injury, cerebral palsy, multiple sclerosis, amyotrophic lateral sclerosis and epilepsy; cardiac conditions, including atherosclerosis, congestive heart failure and myocardial infarction; metabolic disorders, including diabetes; and ocular disorders including macular degeneration and optic atrophy. Such diseases or disorders can be treated either by administration of stem cells themselves, permitting in vivo differentiation to the desired cell type with or without the administration of agents to promote the desired differentiation, or by administering stem cells differentiated to the desired cell type in vitro. Efficacy of treatment is determined by a statistically significant change in one or more indicia of the targeted disease or disorder.

Heart diseases may be treated using the compositions and methods of the invention. In particular, chronic heart failure, myocardial infarction, congestive heart failure, congenital heart disease, cardiomyopathy, pericarditis, angina, and coronary artery disease may be treated using the compositions and methods of the invention.

The compositions of the present invention can be delivered directly or in pharmaceutical compositions, as is known in the art. The present methods involve administration of an effective amount of human stem cells of the present invention to a subject.

An effective amount, e.g., dose, of compositions of the present invention can readily be determined by routine experimentation, as can an effective and convenient route of administration and an appropriate formulation. Various formulations and drug delivery systems are available in the art.

For compositions useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a composition of the present invention refers to an amount or dose of the composition that results in amelioration of symptoms or a prolongation of survival in a subject. Toxicity and therapeutic efficacy of such compositions can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Compositions that exhibit high therapeutic indices are preferred.

The effective amount or therapeutically effective amount is the amount of the composition that will elicit the biological or medical response of a tissue, system, animal, or human that is being sought by the researcher, veterinarian, medical doctor, or other clinician.

### Kits

Another aspect of the invention encompasses kits for treating disease in a subject. A variety of kits having different components are contemplated by the current invention. In some embodiments, the kit will include a TGF-β inhibitor and stem cells. In another embodiment, the kit will include means for collecting a biological sample, means for isolating stem cells from the biological sample, and instructions for use of the kit contents. In certain embodiments, the kit comprises a means for enriching or isolating stem cells in a biological sample. In further aspects, the means for enriching or isolating stem cells comprises reagents necessary to enrich or isolate stem cells from a biological sample. In certain aspects, the kit comprises a means for quantifying the amount of stem cells. In further aspects, the means for quantifying the amount of stem cells comprises reagents necessary to detect the amount of stem cells.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1

### Isolation and Characterization of Cardiac Stem Cells from Rats with Chronic Heart Failure

Cardiac stem cells were isolated from rats that had developed chronic heart failure (CHF) six weeks after myocardial infarction as follows. Two-month-old Sprague Dawley rats (Harlan Laboratories) were anesthetized and ventilated. Following a left thoracotomy, the heart was expressed, and the left anterior descending coronary artery was ligated using a 5-0 TiCron suture. The lungs were briefly hyperinflated, the chest was closed using 2-0 silk, and the rodents were allowed to recover with a pain management regiment of buprenorphine. Sham-operated animals underwent the same surgical procedure excluding left anterior descending artery occlusion.

Hemodynamic statistics were collected from the rats six weeks post-MI using a pressure-volume catheter (Millar Instruments) inserted into the right carotid artery and advanced into the left ventricle. The animals were systemically anesthetized with Inactin (125 mg/kg) and intubated, and the steady-state measurements were collected prior to ventilation. The data were analyzed using PVAN 3.6. software (Millar Instruments).

CHF animals were selected on the basis of left ventricle end-diastolic pressure measurement ≧20 mm Hg and scar size ≧30% of left ventricle. Approximately 35% of infarcted animals were classified as CHF and utilized in subsequent experiments; non-CHF animals were excluded from the study. Atrial tissues were collected six weeks after MI from sham-operated (n=10) and CHF (n=10) animals.

Following collection, atrial tissue was cut into 1- to 2-mm³ pieces and digested with 0.2% trypsin (Life Technologies) and 0.1% collagenase IV (Life Technologies) for a total of 10 minutes. The remaining tissue fragments were cultured as “explants” in explant medium (CEM), which was composed of IMDM supplemented with 10% fetal bovine serum (FBS; Lonza), 100 U/mL penicillin G, 100 μg/mL streptomycin, and 2 mmol/L 1-glutamine (Sigma-Aldrich). After 21 days in culture, cardiac stem cells were collected by trypsinization. c-Kit positive cells were separated from the cell outgrowths using magnetic beads (MACS, Miltenyi Biotec) according to the manufacturer's protocol and analyzed by flow cytometry to validate the purity.

Total RNA was extracted from the cardiac stem cells using PureLink RNA Mini Kit (Life Technologies) according to the manufacturer's protocol. RNA was then quantified with a Quanti-iT RiboGreen RNA Assay Kit and assessed using a BioTek Synergy HT Microplate Reader (excitation/emission 480/520 nm). Total RNA (200 ng) was reverse-transcribed with a QuantiTect Reverse Transcription kit (Qiagen). Real-time RT-PCR was conducted using Rower SYBR Green Master Mix (Applied Biosystems) on a StepOnePlus Real-time PCR System (Applied Biosystems). Specific primers were synthesized by Life Technologies. CYPA was used as a reference gene. Data analysis was performed on StepOne software version 2.1 (Applied Biosystems) using the comparative Ct (AACt) quantitation method.

To assess the amount of TGF-β1 released by explant-derived cells, 0.2×10⁶ cells were cultured for 4 or 10 days, and conditioned media were collected. Cell-culture medium prior to adding cells was also collected to assess baseline levels of TGF-β1. TGF-β1 levels were measured using a commercially available TGF-β1 ELISA kit (R&D Systems) according to the manufacturer's instructions. After conditioned medium was collected, total protein was extracted from cells using RIPA buffer (Thermo Scientific), and the protein amount was determined by a BCA Protein Assay kit (Thermo Scientific). TGF-β1 amounts were normalized to total protein amount.

In another series of experiments, western blotting was carried out as follow. Isolated cardiac stem cells were lysed in RIPA buffer (Thermo Scientific) containing Halt Phosphatase and Proteinase inhibitor cocktail (Thermo Scientific) according to the manufacturer's protocol. Protein concentration was determined using a BCA Protein Assay kit (Thermo Scientific). An equal amount of protein (50 μg) was loaded in each well of 4% to 12% bis-tris gels gel (Life Sciences) and subjected to electrophoresis. Proteins were transferred to a PVDF membrane (Millipore) and then blocked with 5% nonfat dry milk in Tris-buffered saline followed by overnight incubation with primary antibodies at 4° C. Antibodies against p-Smad2/3, Smad2/3 (Cell Signaling), and Nanog (Millipore) were used. Blots were probed with an anti-β-actin (Sigma Aldrich) antibody as a loading control. Membranes were washed in Tris-buffered saline containing 0.05% Tween 20. Corresponding horseradish peroxidase-conjugated anti-rabbit or anti-mouse IgG (Invitrogen) was used as secondary antibodies. Immunoreactive proteins were detected by chemiluminescence (Thermo Scientific). Band intensity was determined using FluorChem 8900 software (Alpha Innotech Corp).

Isolated cardiac stem cells were further characterized using flow cytometry as follows. Cells were fixed in 70% ethanol and labeled with the following antibodies: c-Kit (Santa-Cruz Biotechnology), vimentin and smooth muscle actin (Abcam), and CD90 (BD Biosciences). Cells were treated with secondary antibodies corresponding to either anti-rabbit or anti-mouse IgG conjugated with Alexa 488, phycoerythrin (PE), or PE-Cy5.5 (Life Technologies). Direct labeling with FITC-conjugated CD34 and PE-Cy5.5 conjugated CD45 (BD Biosciences) antibodies was used to exclude bone marrow and hematopoietic cells. Freshly isolated bone marrow cells were used as positive controls for CD34 and CD45 labeling. For a negative control, cells were labeled with isotype IgG instead of primary antibody. Cell events were detected using a FACS Calibur flow cytometer equipped with an argon laser (BD Biosciences). Data were analyzed using CellQuest software (BD Biosciences).

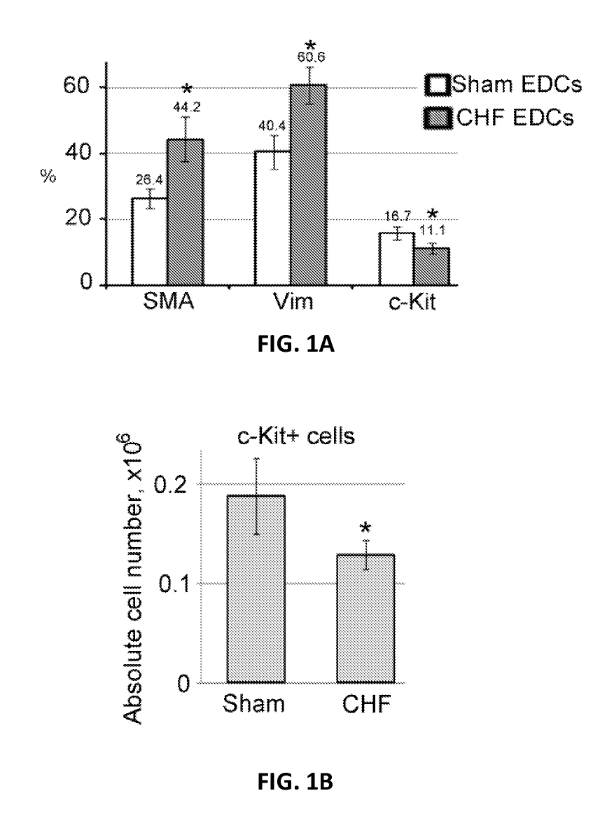

As shown in FIGS. 1A and 1B, compared with sham controls, CHF CSCs comprised a higher number of vimentin-positive fibroblasts and α-smooth muscle actin (SMA)-positive myofibroblasts, and a lower number of c-Kit-positive cells (See FIGS. 1A and 1B).

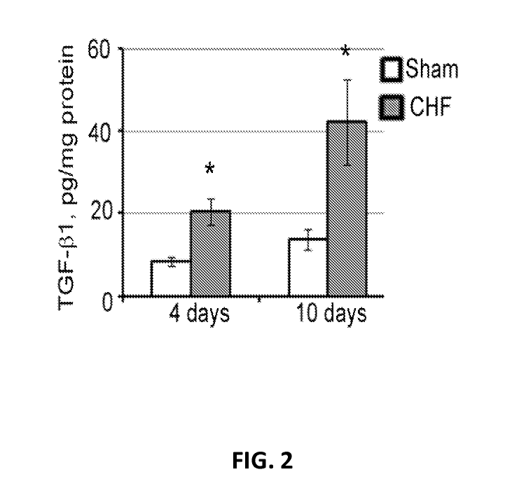

As shown in FIG. 2, CHF stem cells secreted a significantly greater amount of TFG-β1 compared with sham CSCs (see FIG. 2).

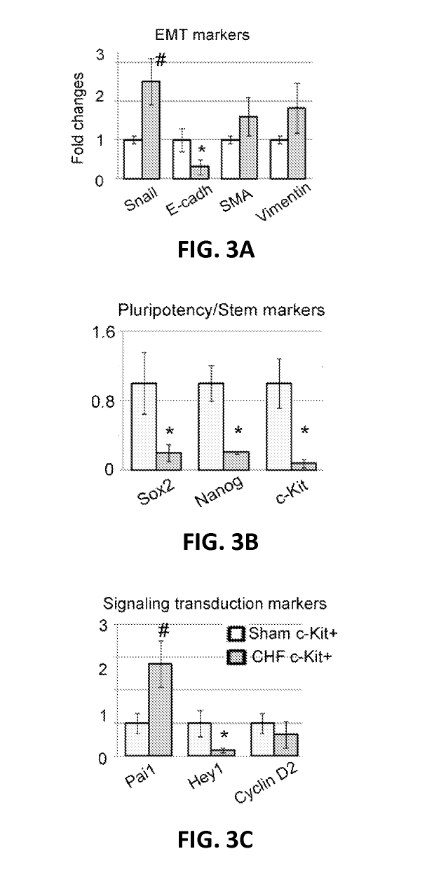

As shown in FIG. 3A, the expression of Snail was 2.5-fold higher in CHF-derived c-Kit+ cells compared with shams. The expression of the epithelial marker E-cadherin, which is negatively regulated by Snail, was 3-fold lower in CHF-derived c-Kit+ cells compared to sham (see FIG. 3A). The expression of vimentin and SMA was increased in CHF-derived c-Kit+ cells compared to sham (see FIG. 3A).

As shown in FIG. 3B, the expression of Sox2 was 4.5-fold lower and Nanog expression was 4.9-fold lower in CHF-derived c-Kit+ cells compared with sham. C-Kit expression was also lower in CHF-derived c-Kit+ cells compared with sham (see FIG. 3B). These results showed that increased TGF-β levels in cardiac stem cells from CHF animals resulted in reduced expression levels of pluripotency markers.

As shown in FIG. 3C, the expression of Pail was 2.8-fold higher in CHF-derived c-Kit+ cells compared with sham. Cyclin D2 expression was similar between CHF-derived c-Kit+ cells and shams. (see FIG. 3C). Hey1 expression was downregulated in CHF c-Kit+ cells compared with shams.

These results showed that cardiac stem cells of the present invention express Nanog, Sox2, c-Kit, Hey1, SMA, Vimenten, Cyclin D2, and E-cadherin but not CD34, CD45, or KDR. These results demonstrated that cardiac stems of the present invention may be isolated and characterized by differential RNA expression. These results showed that cardiac stem cells from CHF animals secrete a greater amount of TGF-β than control cardiac stem cells. The results further showed that methods of the present invention are useful for isolating cardiac stem cells and for selective enrichment of cardiac stem cells. These results further indicated that the methods and cells of the present invention would be useful for isolating cardiac stem cells from a subject suffering from a heart disease.

### Example 2

### TGF-β Inhibition Increased Pluripotency Markers in Cardiac Stem Cells

To determine the effect of inhibition of TGF-β signaling in cardiac stem cells on markers of epithelial/endothelial to mesenchymal transition (i.e., EMT markers) and pluripotency markers the following experiments were performed. Cardiac stem cells were isolated from sham and CHF heart tissue as described in Example 1 above. Cardiac stem cells from CHF and sham animals were treated in culture with TGF-β inhibitors SB431542 or SIS3 for 7 days and subsequently assessed for EMT and pluripotency expression by RNA expression analysis and western blotting using the techniques described above in Example 1.

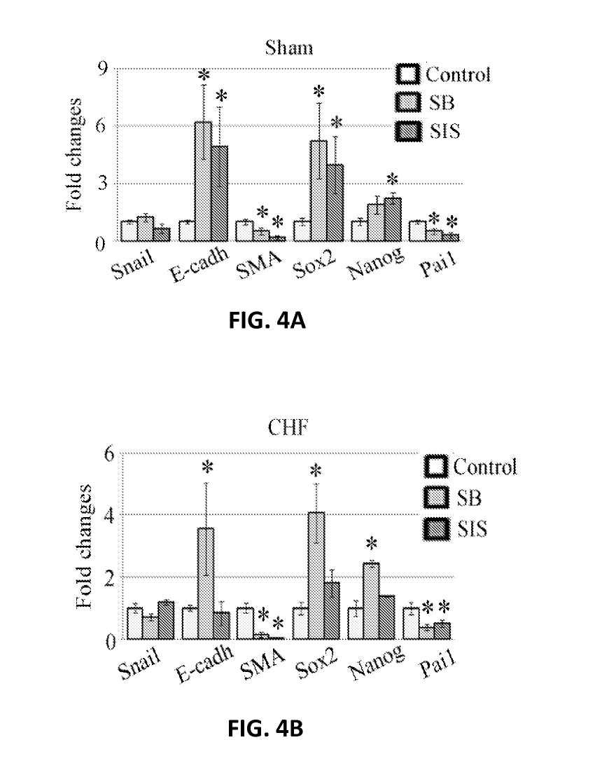

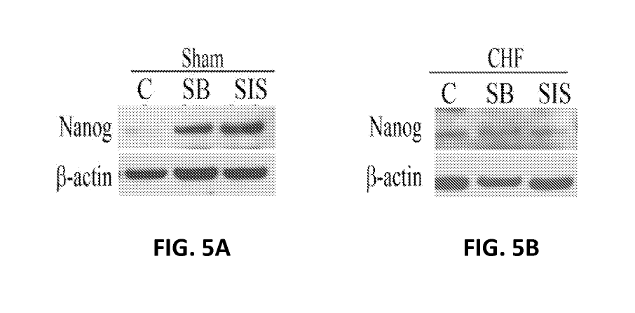

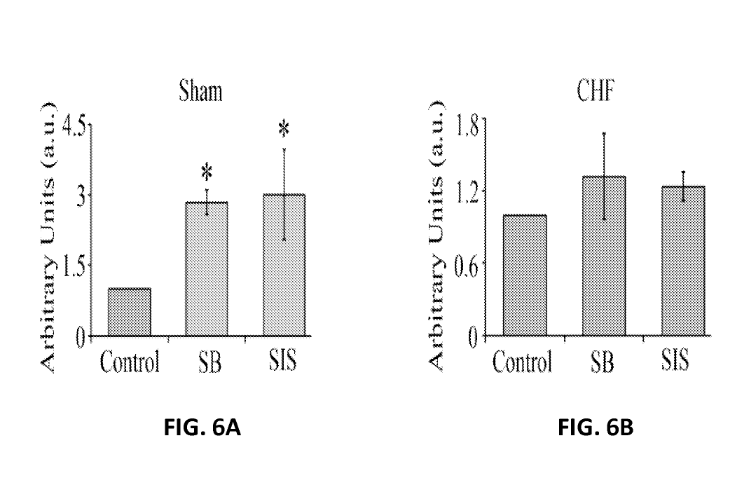

As shown in FIGS. 4A and 4B, in sham cardiac stem cells, both SB431542 and SIS3 reduced SMA gene expression 2- and 5-fold, respectively, whereas in CHF cardiac stem cells, SMA reduction was >10-fold for both inhibitors. Also, SB431542 upregulated E-cadherin expression by 6.2- and 3.6-fold in sham and CHF cardiac stem cells, respectively. SIS3 upregulated E-cadherin expression by 5-fold in sham cardiac stem cells, with no effect on CHF cardiac stem cells. In contrast, SB431542 increased Sox2 expression by 5.2- and 4.1-fold in sham and CHF cardiac stem cells, respectively, whereas SIS3 upregulated Sox2 by 4-fold only in sham cardiac stem cells. Similarly, SB431542 upregulated Nanog expression by 2- and 2.4-fold in sham and CHF cardiac stem cells, respectively, whereas SIS3 upregulated Nanog by 2.2-fold in sham cardiac stem cells only, with no effect in CHF cardiac stem cells. As shown in FIGS. 5A, 5B, 6A, and 6B, TGF-β inhibition increased Nanog protein levels in both sham and CHF cardiac stem cells.

These results showed that TGF-β inhibition increased pluripotency markers in cardiac stem cells from sham and CHF animals. These results further showed that TGF-β inhibitors are useful for increasing pluripotency markers in cardiac stem cells. These results suggest that TGF-β inhibition may be useful for increasing pluripotency of stem cells. These results further suggested that TGF-β inhibition may be useful for stem cell therapy.

### Example 3

### TGF-β Inhibition Increased Differentiation Potential of Cardiac Stem Cells

The effects of TGF-β inhibition on the differentiation potential of cardiac stem cells was examined as follows. Cardiac stem cells were isolated from heart tissue of CHF and sham animals as described in Example 1 above. Cells were subsequently cultured in cardiac differentiation medium (EMD Millipore) supplemented with 2 μmol/L Mocetinostat (SelleckChem) in the presence or absence of TGF-β inhibitors SB431542 and SIS3 for 7 days. Expression of cardiac troponin T (TnT) was evaluated by immunocytochemistry. TnT is an accepted marker for cardiac differentiation. Cells were fixed/permeabilized with a 1:1 acetone:ethanol mixture, blocked with 3% BSA in PBS, and labeled with mouse anti-TnT primary antibody (Abcam). Specific staining was visualized using anti-mouse secondary antibodies conjugated with Alexa 568 (Molecular Probes). Nuclei were stained with 4′,6-diamidino-2-phenylindole (Invitrogen). TnT-positive cells were quantified in 5 random microscopic fields. The percentage of TnT-positive cells was calculated as the number of positively stained cells normalized to the total number of cells.

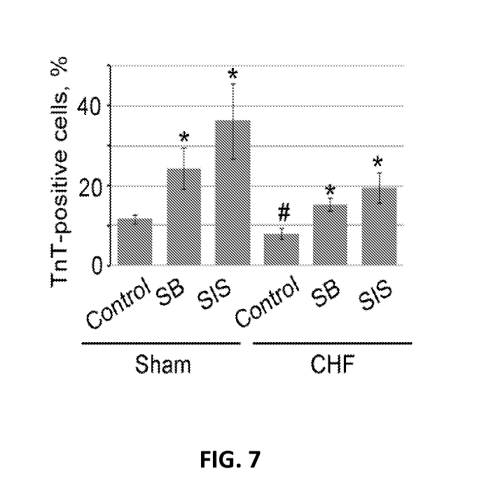

Immunocytochemistry analysis showed higher differentiation potential of sham compared with CHF cardiac stem cells (11.7% versus 8.0% TnT+ cells in sham versus CHF c-Kit+ cells, respectively) (see FIG. 7). As shown in FIG. 7, the presence of SB431542 or SIS3 in culture medium increased the number of TnT-expressing cells in both sham and CHF cardiac stem cells (24.4% and 36.2% TnT+ cells in sham cardiac stem cells treated with SB431542 and SIS3, respectively; 15.4% and 19.6% in CHF cardiac stem cells). These results showed that TGFβ inhibition with SB431542 or SIS3 increases the differentiate potential of cardiac stem cells in vitro. The results further indicated that the methods of the present invention would be useful for increasing the differentiation potential of stem cells. The results suggested that the methods of the present invention may be useful for stem cell therapy.

### Example 4

### TGF-β Inhibition Increased Cardiac Stem Cell Yield from Explants

The effects of TGF-β inhibition on the yield of explant-derived cardiac stem cells were examined as follows. Cardiac stem cells were isolated from heart tissue of CHF and sham animals as described in Example 1 above. Cells were subsequently cultured in cardiac differentiation medium (EMD Millipore) supplemented with 2 μmol/L Mocetinostat (SelleckChem) in the presence or absence of TGF-β inhibitors SB431542 and SIS3 for 21 days. Explant outgrowths were analyzed by flow cytometry as described above in Example 1.

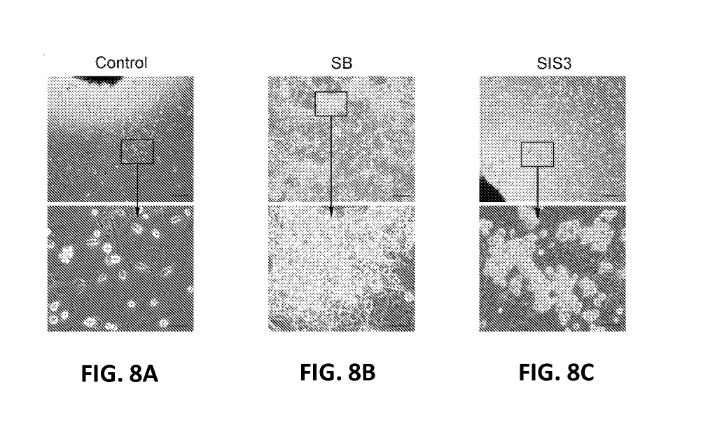

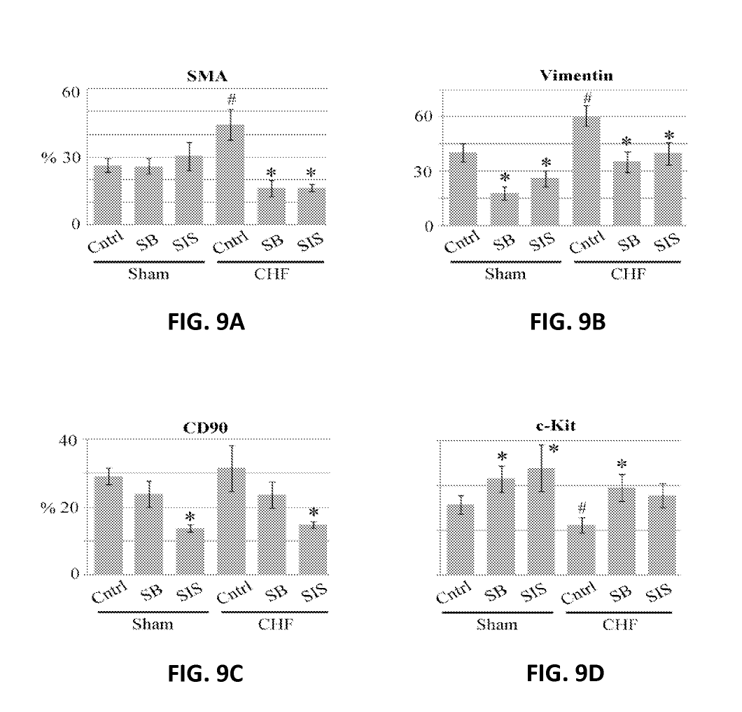

After 21 days in culture, we observed a higher number of small, round phase-bright cell clusters when explants were cultured in the presence of SB431542 and in the presence of SIS3 compared with controls (FIGS. 8A, 8B, and 8C). In addition, when generated in the presence of TGF-β inhibitors, CSCs were smaller in size and appeared tightly connected. Flow cytometry results demonstrated that TGF-β inhibitors decreased the percentage of vimentin-positive fibroblasts in both sham and CHF-derived cardiac stem cells (55% and 35% for SB431542 and SIS3, respectively in sham CSCs; 42% and 34% in CHF CSCs). In CHF CSCs, the number of SMA-positive myofibroblasts was reduced by 63.3% and 62.8% when generated in the presence of SB431542 and SIS3, respectively. In addition, SIS3 reduced the number of CD90-positive cells by 52.4% and 53%, in sham and CHF CSCs, respectively (see FIGS. 9A, 9B, 9C, and 9D). In contrast, SB431542 treatment increased the number of c-Kit+ cells by 36.6% and 75% in sham and CHF CSCs, respectively, whereas SIS3 treatment resulted in a 52% increase in the percentage of c-Kit+ cells in sham CSCs but had no effect on CHF CSCs (see FIGS. 9A, 9B, 9C, and 9D).

These results showed that TGF-β inhibition increased cardiac stem cell yield and function of c-Kit+ cells from sham and CHF cardiac explants. Further, these results showed that TGF-β inhibition enhances the growth rate of cardiac stem cells in culture. These results suggested that the methods of the present invention would be useful for increasing stem cell yield from tissue explants. These results also suggested that TGF-β inhibition would be useful for enhancing the growth rate of stem cells in vitro.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A composition comprising: a human stem cell in admixture with a TGFβ inhibitor.

2. The composition of claim 1, wherein the human stem cell in the admixture is an iPS cell, an embryonic stem cell, a stem cell line, a pluripotent stem cell, a multipotent stem cell, or an adult stem cell.

3. The composition of claim 1, wherein the TGF-β inhibitor is a small molecule, an antibody, or a nucleic acid.

4. The composition of claim 3, wherein the small molecule is SB431542 or SIS3.

5. The composition of claim 1, wherein the human stem cell is a cardiac stem cell.

6. The composition of claim 5, wherein the human stem cell further expresses one or more markers selected from the group consisting of c-Kit, Nanog, Sox2, Hey1, SMA, Vimentin, Cyclin D2, Snail, and E-cadherin, and not one or more of the markers selected from the group consisting of CD34, KDR, and CD45.

7. The composition of claim 1, wherein the human stem cell is obtained from a subject having or suspected of having a heart disease.

8. A method for increasing the pluripotency of a human stem cell comprising treating a human stem cell with an effective amount of a TGF-β inhibitor.

9. The method of claim 8, wherein the TGF-β inhibitor is a small molecule, an antibody, or a nucleic acid.

10. The method of claim 9, wherein the small molecule is SB431542 or SIS3.

11. The method of claim 8, wherein the human stem cell in the admixture is an iPS cell, an embryonic stem cell, a stem cell line, a pluripotent stem cell, a multipotent stem cell, or an adult stem cell.

12. The method of claim 8, wherein the human stem cell is a cardiac stem cell.

13. The method of claim 8, wherein the human stem cell is obtained from a subject having or suspected of having a heart disease.

14. The method of claim 8, wherein the human stem cell further expresses one or more markers selected from the group consisting of c-Kit, Nanog, Sox2, Hey1, SMA, Vimentin, Cyclin D2, Snail, and E-cadherin, and not one or more of the markers selected from the group consisting of CD34, KDR, and CD45.

15. A method of treating a subject suffering from a disease, comprising administering to the subject an effective amount of human stem cells, wherein the human stem cells are pretreated with a TGF-β inhibitor.

16. The method of claim 15, wherein the subject has a heart disease.

17. The method of claim 16, wherein the subject has a heart disease selected from the group consisting of chronic heart failure, myocardial infarction, congestive heart failure, congenital heart disease, cardiomyopathy, pericarditis, angina, and coronary artery disease.

18. The method of claim 15, wherein the human stem cells are iPS cells, embryonic stem cells, a stem cell line, pluripotent stem cells, multipotent stem cells, or adult stem cells.

19. The method of claim 15, wherein the TGF-β inhibitor is a small molecule, an antibody, or a nucleic acid.

20. The method of claim 19, wherein the small molecule is SB431542 or SIS3.

21. The method of claim 15, wherein the human stem cells are cardiac stem cells.

22. The method of claim 15, wherein the human stem cells further expresses one or more markers selected from the group consisting of c-Kit, Nanog, Sox2, Hey1, SMA, Vimentin, Cyclin D2, Snail, and E-cadherin, and not one or more of the markers selected from the group consisting of CD34, KDR, and CD45.
